(19) United States
(12) Patent Application Publication
(10) Pub. No.: US 2023/0172816 A1
(43) Pub. Date: Jun. 8, 2023

(54) COSMETIC COMPOSITION IN AEROSOL FORM, METHOD FOR PRODUCTION AND USE

(71) Applicant: Mariá Aparecida DE LIMA MARTELLI MILAN, Campinas (BR)

(72) Inventor: Mariá Aparecida DE LIMA MARTELLI MILAN, Campinas (BR)

(21) Appl. No.: 17/922,259

(22) PCT Filed: Jul. 23, 2021
(86) PCT No.: PCT/BR2021/050314

§ 371 Date: Oct. 28, 2022

(30) Foreign Application Priority Data
Jul. 29, 2020 (BR) BR1020200154567

(51) Int. Cl. A61K 8/04; A61K 8/34; A61K 8/9789; A61K 8/31; A61Q 19/00

(52) U.S. Cl. CPC A61K 8/046; A61K 8/34; A61K 8/9789; A61K 8/347; A61K 8/31; A61Q 19/00; A61K 2800/522; A61K 2800/10

(57) ABSTRACT

A cosmetic composition in aerosol form is capable of relieving the effects caused by common hormone fluctuations during the climacteric and/or menopause, such as hot flashes, heat waves, excessive heat, night sweats, and excessive sweating. This invention further refers to the method for producing said composition as well as its use for relieving hot flashes, heat waves, excessive heat, night sweats, and excessive sweating in women.

### FIELD OF THE INVENTION

This invention refers to a cosmetic composition in aerosol form capable of relieving the effects caused by common hormone fluctuations during the climacteric and/or menopause, such as hot flashes, heat waves, excessive heat, night sweats, and excessive sweating.

This invention further refers to the method for producing said composition as well as its use for relieving hot flashes, heat waves, excessive heat, night sweats, and excessive sweating in women.

### BACKGROUND OF THE INVENTION

The climacteric is a transitional period in which the woman goes from her reproductive phase to her post-menopause phase. Menopause can thus be defined as the last menstrual period and is usually confirmed when a woman has not had her period for twelve months in a row, a fact that takes place during the climacteric.

During the climacteric, the ovarian functions are reduced, which causes menstrual cycles to become irregular and eventually cease completely. Menopause statistically occurs, in average, at around 50 years of age. The climacteric starts around the age of 40 and extends until the age of 65.

Menopause is defined as the last menstrual period and is usually confirmed when a woman has not had her period for twelve months in a row. Menopause is in itself associated with reduced functioning of ovaries due to the natural aging of the body. This reduction in the function of the ovaries leads to lower levels of estrogen and other natural hormones. This stage marks the permanent end of fertility for women. The symptoms of menopause include heat waves, night sweats, sleep disturbances, vaginal dryness, mood changes, headaches, and irritability.

Heat waves are believed to occur when the nervous cells of the brain hypothalamus, which help regulate the body temperature, erroneously sense that the body is too hot and try to cool it down. The cells in the hypothalamus have estrogen receptors, so, when the estrogen levels go down due to menopause, the body's internal temperature regulators do not function very well, leading to heat waves or night sweating.

The body responds to the signals sent by the nervous cells of the brain hypothalamus, increasing the heart rate and the blood flow toward the edges. As the skin temperature increases due to the higher heart rate and blood flow, the heat and the transpiration mechanism start. When the body temperature falls down due to the cooling effect, the blood vessels contract, and the individual may start to feel cold and humid. When this takes place at night during sleep, it is deemed to be a “night sweat” and usually results in sleep disturbances, fatigue, and irritability.

Every woman is different, but heat waves may occur during menopause at variable levels of intensity. The triggers for heat waves are different in every woman and may include stress, anxiety, certain foods, caffeine, hot beverages, alcohol, warm rooms, or no discernible trigger other than the hormone and chemical fluctuations inside the body.

Therefore, there is a need for some means to alleviate the thermal discomfort caused by menopause.

### SUMMARY OF THE INVENTION

One of the purposes of this invention is a cosmetic composition in aerosol form to relieve hot flashes, heat waves, excessive heat, night sweats, and excessive sweating, encompassing:

a) 0.1% to 0.5% w/w of cooling agents;

b) 0.1% to 0.5% w/w of essential oils;

c) 0.1% to 0.4% w/w of conservatives and/or antioxidants;

d) 50% to 70% w/w of propellants; and

e) 30% to 50% w/w of an alcoholic vehicle;

An additional purpose of this invention is a method to prepare a cosmetic composition in aerosol form covering the stages of:

a) dissolving the cooling agent in a portion of the alcoholic vehicle;

b) incorporating the essential oils;

c) incorporating the conservatives and/or antioxidants;

d) adding the remaining alcoholic vehicle; and

e) inserting it in an adequate recipient and adding the propellant.

An additional purpose of this invention is the use of the cosmetic composition in aerosol form to relieve hot flashes, heat waves, excessive heat, night sweats, and excessive sweating.

These and other advantages will be clearer from the detailed description below.

### DETAILED DESCRIPTION OF THE INVENTION

The following description aims only to exemplify some of the countless ways of using the invention and must not be interpreted as a limitation of the scope of this invention.

Cosmetic Composition in Aerosol Form

The cosmetic composition in aerosol form to relieve hot flashes, heat waves, excessive heat, night sweats, and excessive sweating of this invention encompasses the following ingredients:

a) 0.1% to 0.5% w/w of cooling agents;

b) 0.1% to 0.5% w/w of essential oils;

c) 0.1% to 0.4% w/w of conservatives and/or antioxidants;

d) 50% to 70% w/w of propellants; and

e) 30% to 50% w/w of an alcoholic vehicle;

Cooling agents are agents capable of a providing a feeling of coolness when applied topically. They are present at a concentration ranging from 0.1% to 0.5% w/w, preferably from 0.3% to 0.35% w/w. Examples of cooling agents useful in this invention include, but are not limited to, menthol, menthyl esters, such as menthyl lactate, menthyl succinate and its metallic salts, isopulegol, 3-(1-methoxy)propane-1,2-diol, 3-(1-methoxy)-2-methylpropane-1,2-diol, p-menthane-3,8-diol, p-menthane-2,3-diol, trimethyl cyclohexanol, and combinations thereof. The cooling agent is preferably a combination of menthol and menthyl lactate.

Essential oils are present at a concentration ranging from 0.1% to 0.5% w/w, preferably from 0.2% to 0.3% w/w. Examples of essential oils useful in this invention include, but are not limited to, the Mentha genus, such as Mentha piperita, Mentha pulegium, the Salvia genus, such as Salvia sclarea, Salvia officinalis, Salvia rosmarinus, the Pelargonium genus, such as Pelargonium graveolens, and combinations thereof. The essential oil is preferably a combination of Mentha piperita, Salvia sclarea, and Pelargonium graveolens.

Conservative and/or antioxidant agents are present at a concentration ranging from 0.1% to 0.4% w/w, preferably from 0.1% to 0.3% w/w. Examples of conservatives and/or antioxidants useful in this invention include, but are not limited to, BHA, BHT, phenoxyethanol, DMDM hydantoin, ethylhexylglycerin, and combinations thereof. The conservative and/or antioxidant is preferably a combination of BHT, phenoxyethanol, and ethylhexylglycerin.

Propellants are present at a concentration ranging from 50% to 70% w/w, preferably from 55% to 65% w/w. Examples of propellants include, without limitation, propane, butane, isobutane, dimethyl ether, ethyl fluorides, propyl fluorides, and combinations thereof. The propellant is preferably a combination of propane, butane, and isobutane.

The alcoholic vehicle is present at a concentration ranging from 30% to 50% w/w, preferably from 38% to 42% w/w. It is made up of any alcohol or combination of alcohols useful for a topical application, such as ethanol, and may further include some water content. The alcohol is preferably either ethanol or a grain alcohol.

All other components found in the current state of the art, which are capable of performing some of the abovementioned functions and have not been explicitly mentioned, are part of the scope of the invention, since they are known to an expert in this field, who would have no difficulty at all using them in the above composition.

The above composition must be used to relieve the symptoms of hot flashes and excessive sweats in women.

Composition Production Method

The method to prepare a cosmetic composition in aerosol form according to this invention covers the stages of:

a) dissolving the cooling agent in a portion of the alcoholic vehicle: in case more than a cooling agent is present, they can be dissolved in either a simultaneous or sequential manner, with sequential dissolution being a preferred method. An optimal situation is one that does not use the full content of the alcoholic vehicle for the dissolution. Preferably, the portion used corresponds to ¼ to ⅓ of the total quantity of the vehicle in the composition.

b) incorporating the essential oils: in case more than an essential oil is present, they can be dissolved in either a simultaneous or sequential manner, with sequential dissolution being a preferred method.

c) incorporating the conservatives and/or antioxidants: in case more than a conservative and/or antioxidant is present, they can be dissolved in either a simultaneous or sequential manner, with sequential dissolution being a preferred method.

d) adding the remaining alcoholic vehicle: at this stage, the remaining alcoholic vehicle is added to complete the desired quantity of the water-based vehicle.

e) inserting it in an adequate recipient and adding the propellant: preferably, the adequate recipient is a tin can, to which a valve is installed after the propellant gas is added.

### EXAMPLES

### Example 1—Formulation

Table 1 shows an example of formulation according to this invention:

TABLE 1

| Ingredients | % (w/w) |
|---|---|
| Grain alcohol | 39.2100 |
| Phenoxyethanol, ethylhexylglycerin | 0.2000 |
| Menthol | 0.2000 |
| Menthyl lactate | 0.1200 |
| Mentha piperita essential oil | 0.1200 |
| Sálvia sclarea essential oil | 0.1200 |
| Pelargonium graveolens essential oil | 0.0100 |
| BHT | 0.0200 |
| Butane | 39.0000 |
| Propane | 12.0000 |
| Isobutane | 9.0000 |

An analytical scale was used to weigh the ingredients, and the above formulation was prepared according to the process described below:

a) bulk preparation.

1) The Menthol was weighed and then dissolved in approximately 10 g of grain alcohol;

2) The Menthyl Lactate was weighed and then incorporated into the preceding stage;

3) Each of the essential oils was weighed and then incorporated 1 by 1 to the mixture;

4) The conservative (phenoxyethanol, ethylhexylglycerin) and the antioxidant (BHT) were weighed one by one and then incorporated into the mixture;

5) The remaining grain alcohol was weighed up to q.s.p. 40 g

b) the above bulk was placed in a tin can, and 60 g of propellant gas (butane, propane, isobutane) were added. The valve was placed afterwards.

### Example 2—Stability Test

The samples were stored at 5° C., 25° C., and 40° C. The standard for the Color, Odor, and Appearance analyses was the sample stored at 5° C. The samples were stored at 5° C., 25° C., and 40° C. The standard for the Color, Odor, and Appearance analyses was the sample stored at 5° C. Organoleptic tests—Color, Odor, Appearance (MAFIQ 012), Package Compatibility and Mass Loss (MA FIQ 017), and Microbiology (MA BIO 011/013) tests were conducted, based on Anvisa's Stability Guide, Quality Control Guide for Cosmetics, and USP—NF.

The results are shown in Tables 2 to 7 below:

TABLE 2
Organoleptic Results (appearance) (T0 - A - Slightly
turbid fluid liquid with white particles)

| Time | 5° C. | 25° C. | 40° C. |
|---|---|---|---|
| T0 | A | A | A |
| T30 | A | A | A |
| T60 | A | A | A |
| T90 | A | A | A |

Keys: A—unmodified; B—slight modification; C—acceptable modification; D—unacceptable modification; E—total modification, unrecognizable; NT—not tested; X—not applicable.

TABLE 3
Organoleptic Results (color) (T0 - A - Colorless, slightly whitish)

| Time | 5° C. | 25° C. | 40° C. |
|---|---|---|---|
| T0 | A | A | A |
| T30 | A | A | A |
| T60 | A | A | A |
| T90 | A | A | A |

Keys: A—unmodified; B—slight modification; C—acceptable modification; D—unacceptable modification; E—total modification, unrecognizable; NT—not tested; X—not applicable.

TABLE 4
Organoleptic Results (odor) (T0 - A - characteristic)

| Time | 5° C. | 25° C. | 40° C. |
|---|---|---|---|
| T0 | A | A | A |
| T30 | A | A | A |
| T60 | A | A | A |
| T90 | A | A | A |

Keys: A—unmodified; B—slight loss of fragrance intensity; C—slight loss of fragrance intensity along with a slight modification of the olfactive characteristic; D—sharp loss of fragrance intensity; E—total loss (no fragrance); NT—not tested; X—not applicable.

TABLE 5
Interaction (T0 - A - white metallic bottle, silver bottom,
white plastic actuator, black insert and lid, in a beige
color internally, with particles present)

| Time | 5° C. | 25° C. | 40° C. |
|---|---|---|---|
| T0 | A | A | A |
| T30 | A | A | A |
| T60 | A | A | A |
| T90 | A | A | C |

Keys: A—unmodified; B—slight modification; C—acceptable modification; D—unacceptable modification; E—total modification, unrecognizable; NT—not tested; X—not applicable.

TABLE 6
Mass loss

| | 5° C. | | | 25° C. | | | 40° C. | | |
|---|---|---|---|---|---|---|---|---|---|
| | Sample | Sample | Sample | Sample | Sample | Sample | Sample | Sample | Sample |
| Time | 01 | 02 | 03 | 01 | 02 | 03 | 01 | 02 | 03 |
| T0 | 57.84 | 57.63 | 57.82 | 57.88 | 58.38 | 57.65 | 58.04 | 58.35 | 56.29 |
| T30 | 57.83 | 57.61 | 57.80 | 57.82 | 58.30 | 57.59 | 57.84 | 58.18 | 58.13 |
| T60 | 57.81 | 57.60 | 57.79 | 57.76 | 58.22 | 57.53 | 57.68 | 58.05 | 58.00 |
| T90 | 57.79 | 57.58 | 57.76 | 57.71 | 58.14 | 57.47 | 57.52 | 57.90 | 57.86 |

TABLE 7
Microbiology

| | Total Count | | | Fecal | | |
|---|---|---|---|---|---|---|
| | of Airborne | Total Count | Pseudomonas | Coliforms | Candida | Staphylococcus |
| | Mesophilic | of Mould | aeruginosa | (E. coli) | Albicans | aureus |
| Time | Microorganisms | and Yeasts | (Research) | (Research) | (Research) | (Research) |
| | 5° C. | | | | | |
| T90 | <10 | <10 | Absent | Absent | Absent | Absent |
| | 25° C. | | | | | |
| T0 | <10 | <10 | Absent | Absent | Absent | Absent |
| T90 | <10 | <10 | Absent | Absent | Absent | Absent |
| | 40° C. | | | | | |
| T90 | <10 | <10 | Absent | Absent | Absent | Absent |

A conclusion has been drawn that the product, under study conditions (storage and packaging), keeps its characteristics for the time of the study.

### Example 3—Dermatological Test

A dermatological test assesses primary and accumulated skin irritability and sensitization caused by the test product compared to a control (patch test).

60 individuals were selected, both male and female, aged from 19 to 65, with phototypes (Fitzpatrick) from I to IV.

The informed consent form described the purpose and methodology of the research and was signed by each of the participants.

The product was applied as is over semi-occlusive dressings. Distilled water was used as the control. The dressings were placed on the right or left dorsum of the participants (according to their randomization). The positions of the products and control on each participant's dressings were kept throughout the test.

Initial medical assessment was conducted upon participant enrollment to check for any initial clinical signs incompatible with their enrollment.

The medical assessment data were registered in the investigation book. The physician was available throughout the study to determine any potential adverse events.

The results were assessed as follows:

Discomfort sensations: the participants were inquired about any sensations of discomfort felt, in parallel to their clinical examination. The reported discomfort sensations were described as to their nature (example: burning, stinging, pruritus, twitching, cooling, heating, etc.); were classified as to their intensity as: mild, moderate, or intense; as to their location; and as to their duration, and their association with the test product was verified.

The clinical signs were classified as shown below:

### CLINICAL SIGNS

/—Nothing to report
Ed—Edema
Pu—Pustules
No—Nodes
Cr—Crust
E—Erythema
Pa—Papules
Bo—Blisters
Re—Over-drying/Scaling
V—Vesicle
S—Excessive dryness
C—Coloring (hyperchromia)

### CLASSIFICATION OF CLINICAL SIGNS

Vesicles or papules:
1—No.=1 or 2
2—No.>2
Edema and erythema
1—Mild
2—Moderate
3—Severe/intense
Appearance of the erythema and edema
d—Diffuse
p—Punctate
peri—Peripheral

A patch test was employed. The product and the control were applied over a semi-occlusive dressing on the dorsum of the participant, on the left or on the right (according to the randomization).

The patch test remained on the skin for 48 hours, after which it was removed for the clinical signs to be read and for inquiry on the discomfort sensations by the dermatologist. After the reading, a new dressing was placed, keeping the products in the same position.

All readings were registered in the investigation book. The applications were performed following Table 8 below:

TABLE 8
Application and reading of the composition

| | | Day of the Week | | | | |
|---|---|---|---|---|---|---|
| | Week | Monday | Tuesday | Wednesday | Thursday | Friday |
| Induction | 1st | A | | A + L | | A + L |
| Phase | 2nd | A + L | | A + L | | A + L |
| | 3rd | A + L | | A + L | | L |
| Resting | 4th | No dressing applied | | | | |
| Phase | 5th | | | | | |
| Challenge | 6th | A + L | | L | | |
| Phase | | | | | | |

Keys: A = application; L = reading

Of all 60 initial participants, 54 completed the study. No adverse effects were detected on the areas where the product and the control were applied during the period of the study. No participant reported a sensation of discomfort from the product or the control during the study.

1. A cosmetic composition in aerosol form comprising:
a) 0.1% to 0.5% w/w of cooling agents;
b) 0.1% to 0.5% w/w of essential oils;
c) 0.1% to 0.4% w/w of conservatives and/or antioxidants;
d) 50% to 70% w/w of propellants; and
e) 30% to 50% w/w of an alcoholic vehicle;

2. The composition, as per claim 1, wherein the cooling agents are chosen from the group consisting of menthol, menthyl esters, such as menthyl lactate, menthyl succinate and its metallic salts, isopulegol, 3-(1-methoxy)propane-1,2-diol, 3-(1-methoxy)-2-methylpropane-1,2-diol, p-menthane-3,8-diol, p-menthane-2,3-diol, trimethyl cyclohexanol, and combinations thereof.

3. The composition, as per claim 1, wherein the essential oils are chosen from the group consisting of the oils Mentha piperita, Mentha pulegium, Salvia sclarea, Salvia officinalis, Salvia rosmarinus, Pelargonium graveolens, and combinations thereof.

4. The composition, as per claim 1, wherein the conservatives and/or oxidants are chosen from the group consisting of BHA, BHT, phenoxyethanol. DMDM hydantoin, ethylhexylglycerin, and combinations thereof.

5. The composition as per claim 1, wherein the propellants are chosen from the group consisting of propane, butane, isobutane, dimethyl ether, ethyl fluorides, propyl fluorides, and combinations thereof.

6. The composition, as per claim 1, wherein the alcoholic vehicle is chosen between ethanol or grain alcohol.

7. The composition, as per claim 1, wherein the composition includes the following ingredients:

| Ingredients | % (w/w) |
|---|---|
| Grain alcohol | 39.2100 |
| Phenoxyethanol, ethylhexylglycerin | 0.2000 |
| Menthol | 0.2000 |
| Menthyl lactate | 0.1200 |
| Mentha piperita essential oil | 0.1200 |
| Sálvia sclarea essential oil | 0.1200 |
| Pelargonium graveolens essential oil | 0.0100 |
| BHT | 0.0200 |
| Butane | 39.0000 |
| Propane | 12.0000 |
| Isobutane | 9.0000 |

8. A method for producing an aerosol composition as defined in claim 1, wherein the method covers the stages of:
a) dissolving the cooling agent in a portion of the alcoholic vehicle;
b) incorporating the essential oils;
c) incorporating the conservatives and/or antioxidants;
d) adding the remaining alcoholic vehicle; and
e) inserting it in an adequate recipient and adding the propellant.

9. The method, as per claim 8, wherein the dissolution and/or incorporation of the ingredients in stages a), b), and/or c) is simultaneous or sequential.

10. The method, as per claim 9, wherein the dissolution and/or incorporation of the ingredients in stages a), b), and/or c) is sequential.

11. The method, as per claim 8, wherein the method encompasses an additional stage of placing a valve onto the recipient after adding the propellant.

12. A use of a composition in aerosol form defined in claim 1 wherein the method aims to relieve hot flashes, heat waves, excessive heat, night sweats, and excessive sweating in women.
